(19) United States
(12) Patent Application Publication
(10) Pub. No.: US 2023/0115937 A1
(43) Pub. Date: Apr. 13, 2023

(54) NEOPHALLUS NEOTUNICA IMPLANT FOR NEOPHALLUSES

(71) Applicant: MHN BIOTECH LLC, Chevy Chase, MD (US)

(72) Inventors: Arthur L. BURNETT, Baltimore, MD (US); Howard S. NEWMAN, Annapolis, MD (US); Marcel I. HOROWITZ, Chevy Chase, MD (US)

(73) Assignee: MHN BIOTECH LLC, Chevy Chase, MD (US)

(21) Appl. No.: 17/498,497

(22) Filed: Oct. 11, 2021

(51) Int. Cl. A61F 2/26; A61F 2/00

(52) U.S. Cl. CPC A61F 2/26; A61F 2/0077; A61F 2230/0069; A61F 2250/006; A61F 2250/0071

(57) ABSTRACT

The present disclosure is directed to a system and method for containing a penile prosthesis in a neophallus, to stabilize this device in the neophallus and to give the neophallus additional stiffness in erection. The contained penile prosthesis further resembles a glans at the distal end of the neophallus.

### FIELD OF THE INVENTION

The present disclosure relates to the field of implanted medical devices. More particularly, the present disclosure relates to systems and methods in penile reassignment and reconstruction surgery wherein a “neophallus neotunica” is implanted within the neophallus to stabilize a penile prosthesis placed in the neophallus to facilitate erection. Furthermore, the neophallus neotunica implant provides structural support, stiffness and anatomical shape and feel to the neophallus.

### BACKGROUND OF THE INVENTION

Penile prostheses have been available for decades to treat erectile dysfunction. More recently, they have been used in penile reconstruction surgery to provide the new penis, or neophallus, with erectile capability. For example, clinicians are developing surgical procedures and devices to treat patients with severe penile defects or loss. Examples of such defects and loss include congenital penile abnormalities (e.g., aphallia, micropenis, complex severe hypospadias, classic bladder exstrophy/epispadias complex, cloacal exstrophy), sexual differentiation disorders (i.e., ambiguous genitalia), penile trauma resulting in penile destruction, penectomy (e.g., as required for treating malignancy), and female-to-male gender reassignment.

These conditions may be addressed with major genital reassignment and reconstructive procedures using local tissue or autogenous reconstruction (i.e., using tissue flaps) of a phallus or penile allotransplantation. In these cases, a penile prosthesis is commonly inserted into the neophallus to facilitate erection. Most often, either single or double inflatable cylinders prostheses are implanted. Alternatively employed are semi-rigid or heat sensitive (malleable) rods which bend into an erect position. Since these rods maintain length, they can only be bent away when no erection is desired.

In a typical procedure using an inflatable cylinder prosthesis, the inflatable cylinder has a distal end in the form of a solid cone with a small hole drilled through it to accept a suture. A Furlow tool and Keith needle are usually used to pull the prosthesis distally into the neophallus. For implanted rods, the stiffness of the rod allows direct insertion into the neophallus. In both cases, the neophallus takes the shape of the penile prosthesis' distal end, which does not present as a phallus with glans.

Furthermore, there is currently no technique routinely prescribed to prevent the neophallus from stretching out distally, leaving no support for the neophallus' distal end. This prosthesis malpositioning, the “floppy glans syndrome,” is frequently observed and often requires reoperation. Additionally, in these procedures, there is the common disturbance of palpating the penile prosthesis' distal end just beneath the skin of the distal neophallus; or, worse, its erosion distally out the end of the neophallus. Also, in erection, the stiffness of an inserted inflatable penile prosthesis after inflation may be reduced in a neophallus relative to that of a normal phallus.

While procedures such as directly attaching a glans-like shape to the distal end of an inflatable penile prosthesis and neotunica grafting are known, these procedures do not address both problems of penile implant movement and instability within the neophallus and neophallus shape and feel, which have caused a large number of implants to be removed at a later date due to erosion, migration, or infection. Patients are often displeased with any or all of the complications described above.

What is needed, in the context of a penile prosthesis device inserted within a neophallus, is a device that will contain and secure in place a penile prosthesis; prevent the migration of the penile prosthesis cylinder proximally or distally relative to the dimensions of the neophallus; frame the neophallus into the shape and feel of a normal phallus; and create stiffness in erection that mimics that of a normal phallus.

### SUMMARY OF THE INVENTION

The present invention is directed to systems and methods for a neophallus neotunica structural integrity and shape implant device. Specifically, the systems and methods relate to a neophallus neotunica structural integrity and shape device for implantation into a neophallus. The neophallus neotunica ensheathes and secures at least one penile prosthesis cylinder interior to it while its exterior allows fatty tissue from the neophallus to adhere to it thereby stabilizing it within the neophallus. The systems and methods prevent migration and other adverse sequelae of an unbefitting penile prosthesis inserted in a neophallus. Moreover, the systems and methods give the neophallus the shape and feel of a normal phallus and provide the neophallus in erection the stiffness of a normal phallus.

The neophallus neotunica is comprised of a “neo-tunica albuginea” in communication with a “neo-glans.” The neo-tunica albuginea functions as the tunica albuginea that naturally surrounds and supports the corporal bodies of the native penile shaft. It may ensheathe at least one penile prosthesis cylinder. For inflatable penile prostheses, it provides the neophallus with support and additional stiffness in erection and stabilizes the distal and proximal ends of the penile prosthesis in the neo-tunica albuginea.

The neo-glans is in communication with the neo-tunica albuginea distally. It approximates the shape and feel of an anatomical glans and provides at least one socket to fasten the distal end of at least one penile prothesis cylinder. For inflatable penile prostheses, the socket may secure in place at least one penile prosthesis cylinder to prevent its migration proximally or distally with risk of erosion through the distal end of the neophallus. The socket may be encased in a soft material designed to approximate the shape and feel of an anatomical glans.

For inflatable penile prostheses, the neophallus neotunica may comprise a cylindrical neo-tunica albuginea in the form of a sock, wherein its closed, distal end may ensheathe a neo-glans, and wherein its proximal extent may ensheathe at least one penile prosthesis cylinder. The neo-tunica albuginea's sock may have a framework with external concentric ridges along its length to affix it in place during implantation; it may be comprised of nonabsorbent surgical mesh that will adhere within the subcutaneous fatty tissue of the neophallus shaft postoperatively as it becomes infiltrated by fat, thereby affixing it in place; it may be comprised of acellular dermal matrix material; or it may be comprised of thin-wall tubing.

For inflatable penile prostheses, the neophallus neotunica may also comprise a cylindrical neo-tunica albuginea whose distal end may be in communication with a neo-glans, and wherein its proximal extent may ensheathe at least one penile prosthesis cylinder. It may have a framework of external concentric ridges along its length to affix it in place during implantation; it may be comprised of nonabsorbent surgical mesh that will adhere within the subcutaneous fatty tissue of the neophallus shaft postoperatively as it become infiltrated by fat, thereby affixing it in place; it may be comprised of a cellular dermal matrix material; or it may be comprised of thin-wall tubing.

To increase stiffness of the inflatable penile prostheses during inflation for erection, the neo-tunica albuginea may be comprised of finitely expansile inner and outer tubes, with the outer tube having an outside diameter that approximates the maximal girth of the inflated penile prosthesis cylinder. Columns of small, thin, overlapping disks, which may be circular, elliptical or petaled, may be suspended in a soft matrix material between the two tubes. When the inflatable penile prothesis is flaccid, the disks are free to move with respect to each other allowing the neophallus to droop. When the inflatable penile prosthesis is inflated, the thin disks are opposed together between the inner and outer tubes, causing the structure to stiffen.

For semi-rigid rod (malleable) penile prostheses, the neophallus neotunica may comprise a cylindrical neo-tunica albuginea in the form of a sock, wherein its closed, distal end may ensheathe a neo-glans, and wherein its framework proximally may ensheathe at least one semi-rigid penile prosthesis cylinder.

The neo-glans approximates the shape and feel of an anatomical glans and provides at least one socket to house the distal end of at least one semi-rigid rod. The socket may be encased in a soft material to approximate the shape and feel of an anatomical glans.

The sock may be comprised of material such as spandex whose nominal diameter is less than that of the semi-rigid rod. When the semi-rigid rod is inserted within the sock, the expanding spandex will exert pressure on the semi-rigid rod and neo-glans to keep them in place within the sock.

The sock may have a framework with external concentric ridges along its length externally to affix it in place during implantation; it may be comprised of an open-weave mesh that will adhere within the subcutaneous fatty tissue of the neophallus shaft postoperatively as it become infiltrated by fat, thereby affixing it in place; or it may be comprised of thin-weave tubing.

The neophallus neotunica may be comprised of FDA approved implantable material such as silicone, polypropylene, PVDF, or a combination there of.

The neophallus neotunica with its ensheathed penile prosthesis may be inserted in the neophallus in a late stage of penile reconstruction surgery.

### BRIEF DESCRIPTION OF THE DRAWINGS

The foregoing aspects, features, and advantages of the present disclosure will be further appreciated when considered with reference to the following description of embodiments and accompanying drawings. In describing the embodiments of the disclosure illustrated in the appended drawings, specific terminology will be used for the sake of clarity. However, the disclosure is not intended to be limited to the specific terms used, and it is to be understood that each specific term includes equivalents that operate in a similar manner to accomplish a similar purpose.

FIG. 1A is an illustration of a vertical cross-section along the principal axis of one cylinder of an inflatable penile prosthesis.

FIG. 1B is an illustration of vertical cross-section along the principal axis of one cylinder of a flaccid semi-rigid cylinder penile prosthesis.

FIG. 2A is an illustration of a vertical cross-section along the principal axis of a neo-glans with the distal end of an inflatable penile prothesis cylinder captured in its socket catch.

FIG. 2B is an illustration of a vertical cross-section along the principal axis of a neo-glans with the distal end of a semi-rigid rod penile prothesis cylinder held in its socket.

FIG. 3A is an illustration of a vertical cross-section along the principal axis of a neo-tunica albuginea sock ensheathing a neo-glans and one cylinder of an inflatable penile prosthesis.

FIG. 3B is an illustration of a vertical cross-section along the principal axis of a neo-tunica albuginea with one cylinder of an inflatable penile prosthesis ensheathed and with a neo-glans in communication at its distal end.

FIG. 4A is a side view of the proximal end of an inflatable penile prosthesis ensheathed in a neo-tunica albuginea with two elastic bands holding it in its place in the neo-tunica albuginea.

FIG. 4B is a side view of a semi-rigid rod prosthesis inserted into an elastic neo-tunica albuginea.

FIG. 5A is an illustration of a vertical cross section along the minor axis of a neophallus neotunica ensheathing a deflated inflatable penile prosthesis. Small, thin, overlapping disks placed between the inner and outer cylinders of the neophallus neotunica are unaligned during deflation allowing the neophallus to droop.

FIG. 5B is an illustration of a vertical cross section along the minor axis of a neophallus neotunica ensheathing an inflated inflatable penile prosthesis. Small, thin, overlapping disks placed between the inner and outer cylinders of the neophallus neotunica become aligned during inflation to stiffen the neophallus.

FIG. 6 is an illustration of a vertical cross section along the principal axis of a neophallus neotunica ensheathing a dual cylinder inflatable penile prosthesis and attached to a dual socket neo-glans.

### DETAILED DESCRIPTION OF THE INVENTION

Embodiments of the present disclosure relate to systems and methods involving a neophallus neotunica device which may be inserted into a surgically reconstructed penis, a neophallus, to contain and secure a penile prosthesis inserted in a neophallus, which provides the structure for the neophallus to produce a satisfactory erection. Furthermore, the neophallus neotunica provides structural support, stiffness and anatomical shape and feel to the neophallus.

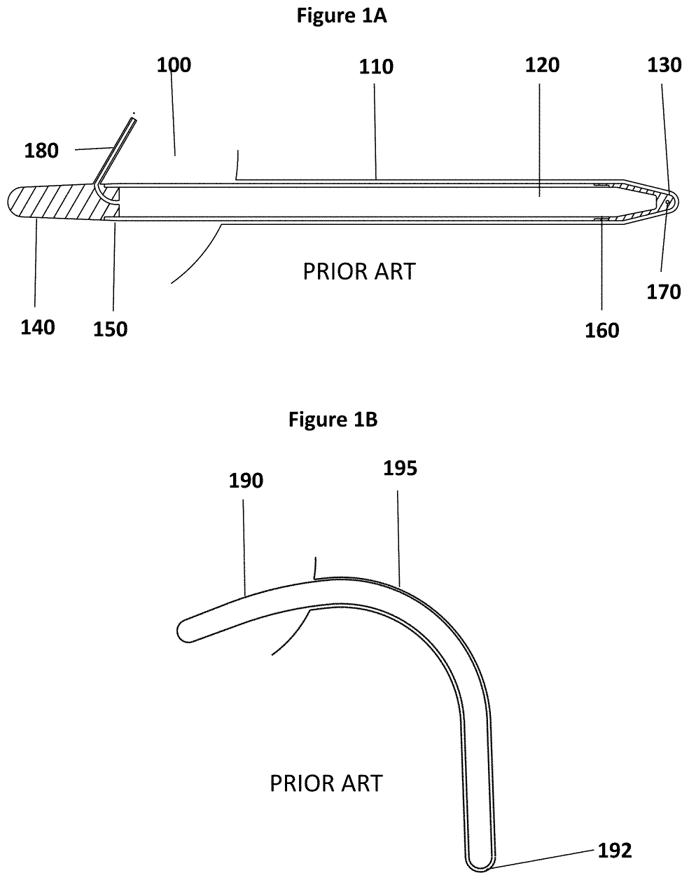

FIG. 1A illustrates a typical inflatable penile prosthesis cylinder 100 currently used in penile reconstruction surgery to provide erectile capability in a neophallus 110. As shown in FIG. 1A, the penile prosthesis cylinder 100 includes an inflatable cylinder 120 in communication with a solid distal endcap 130 and a solid proximal endcap 140. The inflatable cylinder 120 is inflated and deflated by isotonic fluid which is pumped into and out of the inflatable cylinder through an exit tubing 180.

The solid proximal endcap 140 seals the proximal end of the inflatable cylinder and provides a foundation for attaching the penile prosthesis 100 to the pelvis so it does not move distally or proximally during sexual intercourse. The distal endcap 130 seals the distal end of the inflatable cylinder 120 and has a hole 170 drilled through it to accept a suture which may be used to pull the inflatable penile implant 100 into a phallus or neophallus 110 during implantation. Waterproof joints 150, 160 may use adhesives and heat shrinking sleeves to connect the endcaps 130, 140 to the inflatable cylinder 120.

FIG. 1B illustrates a typical semi-rigid rod prosthesis implant 190 in flaccid position that has been previously used in penile reconstruction surgery to provide a phallus or neophallus 195 with erectile capability. The semi-rigid rod's 190 shape is moved up and down manually by the patient into erect or flaccid position.

As illustrated in FIGS. 1A and 1B, after implantation, the distal end of the neophallus 110, 195 takes the shape of the penile prostheses' 100, 190 distal end 130, 192 which do not appear as anatomically correct glans. Once implanted, there is nothing connecting the penile prostheses 100, 190 to the neophallus 110, 195 and the neophallus may elongate causing its distal end to droop, or it may wear away at the distal end of the penile prosthesis cylinder 130, 192 causing it to erode through the neophallus 110, 195. Additionally, inflatable penile prostheses do not provide the neophallus with the same degree of stiffness in erection as a normal phallus.

In contrast to directly placing at least one existing penile prosthesis cylinder, shown in FIGS. 1A and 1B and described above, directly into a neophallus, the present invention is directed towards systems and methods for surgeons to first ensheathe at least one penile prosthesis cylinder in a neophallus neotunica and then insert the neophallus neotunica into the neophallus thereby stabilizing the penile prosthesis cylinder in its place and preventing its malposition in the neophallus and also giving the neophallus the look and feel of a native phallus.

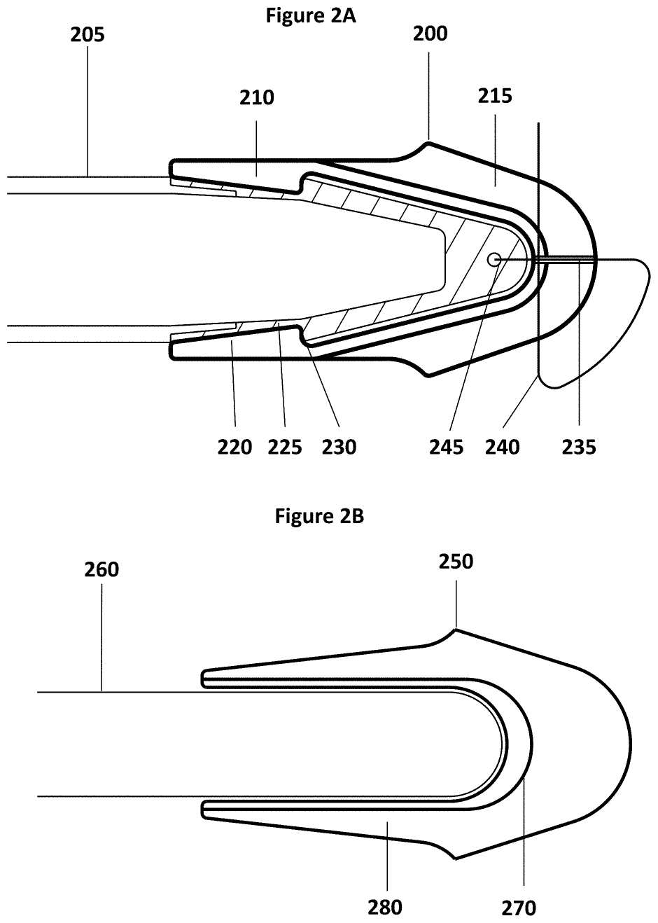

In an embodiment, the neophallus neotunica may be comprised of a neo-glans in communication with a neo-tunica albuginea. FIG. 2A illustrates a neo-glans 200 in communication with the distal end of an inflatable penile prosthesis 205. The neo-glans 200 is comprised of at least one socket catch 210 which is ensheathed in a softer material 215, approximating the shape and feel of an anatomical glans. The socket catch 210 is further comprised of ramp 220 which compresses the inflatable penile prosthesis' solid distal endcap 225 upon entry and allows it to expand back out after being pulled inside the socket clamp 210. An edge 230 of the ramp 220 prevents the expanded solid distal endcap 225 from being pulling back out during normal use.

A hole 235 in the neo-glans 200 may be provided for a suture 240, emanating from a hole 245 in inflatable penile implant 205, to pull the inflatable penile implant 205 into the neo-glans' 200 socket catch 210. The suture 240 may then be threaded back through the socket catch 210 to further secure the inflatable penile prothesis 205 in the neo-glans 200.

The surgeon may be able to extract the penile prosthesis 205 from the neo-glans 200 by cutting the suture 240 and pulling the inflatable penile prosthesis 205 out of the neo-glans 200 should repair or removal be necessary. The neo-glans 200 may come in different sizes to meet different patients' body sizes and neophallus sizes.

FIG. 2B illustrates another embodiment of a neo-glans 250 in communication with the distal end of a semi-rigid penile prosthesis cylinder 260. The neo-glans 250 is comprised of at least one socket 270 which is ensheathed in a softer material 280, approximating the shape and feel of an anatomical glans. The socket 270 may be slightly smaller than the distal end of the penile prosthesis cylinder compressing it to keep it in place,

The neo-glans may be made from FDA approved plastic material, such as silicone, with the socket designed to be stiffer than its ensheathing material.

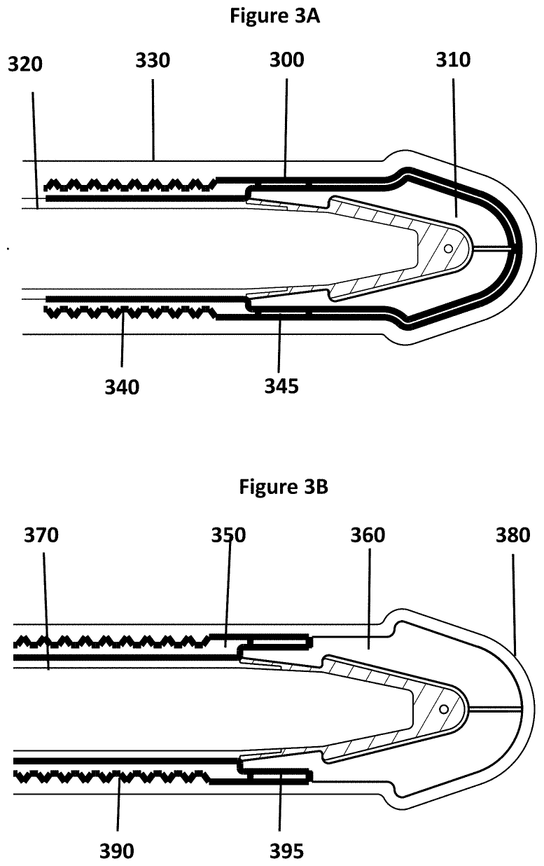

FIG. 3A illustrates an embodiment of the distal end of a neo-tunica albuginea in the form of a flexible sock 300, wherein its closed, distal end ensheathes a neo-glans 310, and wherein its open proximal end ensheathes an inflatable penile prosthesis cylinder 320. In this embodiment, the sock 300 may be in the form of a cylinder with its external surface having concentric ridges 340 to affix it in place once it is pulled up into the dilated space inside the neophallus 330. The sock 300 may be comprised of silicone or polyethylene.

The sock 300 may also be in the form of nonabsorbent surgical mesh that will adhere within the subcutaneous fatty tissue of the neophallus shaft postoperatively as it become infiltrated by fat, thereby affixing it in place. Alternatively, the sock 300 may be constructed in the form of an acellular dermal matrix material, or as a smooth thin wall sock consisting of materials such as silicone or polyethylene.

A joint 345 may be provided to affix the neo-glans 310 to the sock 300. For example, the joint 345 may use a biocompatible adhesive, such as epoxy, to affix the neo-glans to the sock. The sock 300 may be constructed in various sizes. The surgeon may select the required size and trim it to length during implantation.

FIG. 3B illustrates the distal end of another embodiment of a neo-tunica albuginea in the form of a flexible tube 350 whose distal end is in communication with a neo-glans 360 and wherein its proximal end ensheathes an inflatable penile prosthesis 370. In this embodiment, the tube's external surface may have concentric ridges 390 to affix it in place once it is pulled up into the dilated space inside the neophallus 380. The tube 300 may be comprised of silicone or polyethylene.

The tube 350 may also be in the form of nonabsorbent surgical mesh that will adhere within the subcutaneous fatty tissue of the neophallus shaft postoperatively as it become infiltrated by fat, thereby securing it in place. Alternatively, the tube 350 may be constructed in the form of an acellular dermal matrix material or as a smooth, thin wall tubing consisting of materials such as silicone or polyethylene.

A joint 395 may be provided to affix the neo-glans 360 to the tube 350. For example, the joint 395 may use a biocompatible adhesive, such as epoxy, to affix the neo-glans to the tube. The tube 350 may be constructed in various sizes. The surgeon may select the required size and trim it to length during implantation.

To further secure penile prostheses cylinders in the neophallus neotunica, elastic material may be used to apply pressure on the neo-tunica albuginea pressing it against hard surfaces in the penile prosthesis cylinder.

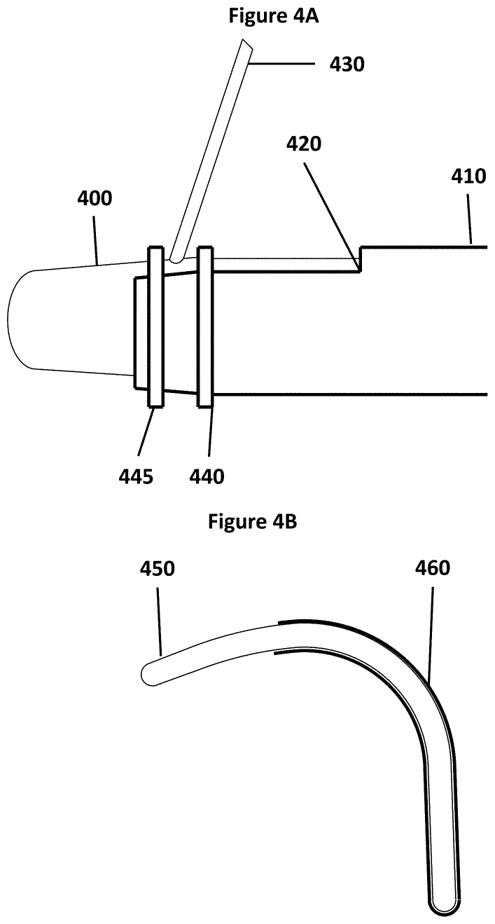

FIG. 4A illustrates the proximal hard endcap 400 of an inflatable penile prosthesis cylinder ensheathed by a neo-tunica albuginea 410. A cutout 420 is made in the neo-tunica albuginea 410 to allow it to extend beyond the inflatable penile prosthesis' exit tubing 430. Elastic bands 440, 445 are then placed distally and proximally around the ensheathed cylinder to secure the neo-tunica albuginea to the cylinder preventing distal and proximal movement of the proximal hard endcap 400 within the neo-tunica albuginea 410.

FIG. 4B illustrates a semi-rigid penile prosthesis cylinder 450 ensheathed in an elastic sock neo-tunica albuginea 460. The elastic sock 460 puts pressure on the semi-rigid penile prosthesis 450 keeping it in place in the neo-tunica albuginea. In an embodiment, the elastic sock may be comprised of a stretch material such as Spandex, which may be in the form of a cylinder with its external surface weave having concentric ridges to affix it in place once it is pulled up into the dilated space inside a neophallus. In another embodiment, the sock 460 may be in the form of a loose weave elastic mesh that will adhere within the subcutaneous fatty tissue of the neophallus shaft postoperatively as it become infiltrated by fat, thereby securing it in place.

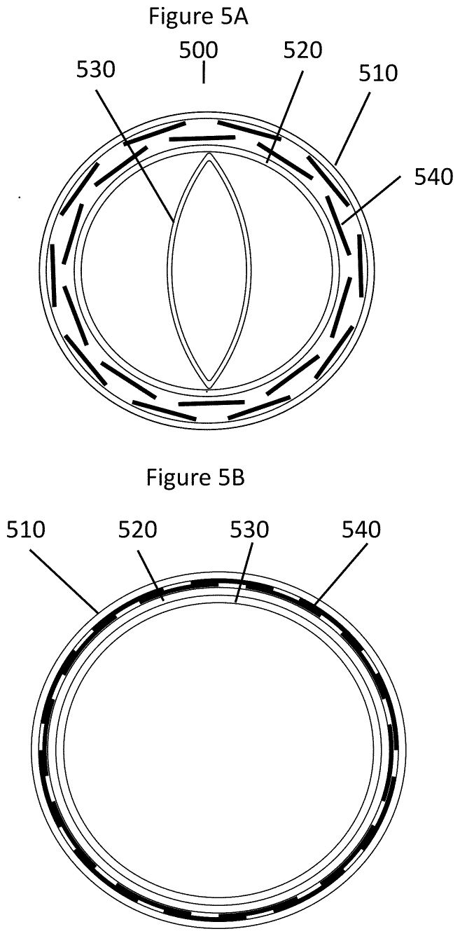

Some patients are not satisfied with the stiffness in erection of existing inflatable penile prostheses placed in neophalluses. To address this problem, the neophallus neotunica of the present invention may contain an apparatus that that will stiffen in erection and allow drooping when flaccid, as described in FIGS. 5A and 5B.

FIG. 5A illustrates a cross section view of a neo-tunica albuginea 500, designated by its outer cylinder 510 and an inner cylinder 520 ensheathing a deflated inflatable penile prosthesis cylinder 530. The inner cylinder of the neo-tunica albuginea 520 is external to the inflatable penile prosthesis cylinder 530. The space between the inner cylinder 520 and the outer cylinder 510 may contain columns of small, thin, overlapping sequin-like disks 540 which may be circular, elliptical, or four-leaf clover shaped. The disk columns 540 may be supported in this space by a loosely woven fabric structure or embedded in a spongy material which may be made from silicone. When the inflatable penile prosthesis 530 is deflated the disks 540 are unaligned with respect to each other allowing the neophallus to droop.

FIG. 5B illustrates a cross section view of the neo-tunica albuginea, designated by its outer cylinder 510 and an inner cylinder 520 ensheathing an inflated inflatable penile prosthesis cylinder 530. When the inflatable penile prosthesis cylinder 530 is inflated, it expands to compress the inner cylinder 520 against the outer cylinder 510 of the neo-tunica albuginea forcing the disks 540 to compress. Each disk's 540 lateral dimension produces a semi-cylindrical curve, strengthening it along its longitudinal dimension and aligning it with other disks thereby providing stiffness to the neophallus in erection.

Some patients request implantation of dual penile prosthesis cylinders to match the two corpus cavernosum of an anatomical penis. In penile prosthesis implant surgery, dual penile prothesis cylinders are usually anchored medially to the bilateral ischia of the pelvis. Distally, they must align together medially in the neophallus without crossing each other or twisting.

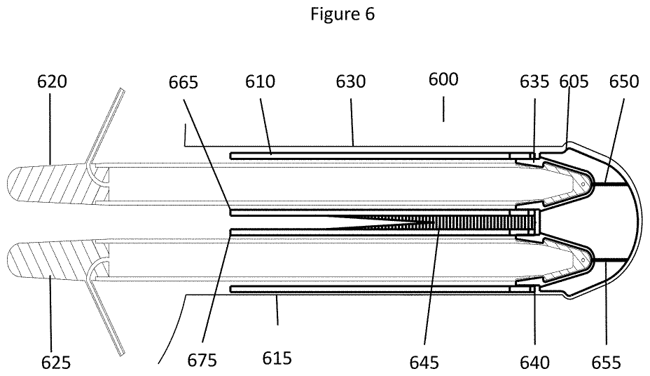

FIG. 6 illustrates a neophallus neotunica 600 for a dual cylinder penile prosthesis. It is comprised of a dual cylinder neo-glans 605 in communication with two neo-tunica albuginea 610, 615 for ensheathing two penile prosthesis cylinders 620, 625 allowing them to be anchored separately in the pelvis while maintaining their parallel position in the neophallus 630. The dual neo-glans 605 may have two socket catches 635, 640, such as those illustrated in FIG. 2A. The dual neo-tunica albuginea 610 615 may resemble one of the neo-tunica albuginea illustrated in FIGS. 3A and 3B.

A neotunica albuginea septum 645 which may be comprised of unabsorbant surgical mesh or acellular dermal matrix material and may connect the dual neo tunica albuginea 610, 615. The neo-tunica albuginea septum 645 may be separated and trimmed at its proximal end 665, 675 allowing the two penile prosthesis cylinders 620, 625 to be aligned with and attached to each ischium of the pelvis.

Two holes 650, 655 in the neo-glans 605 may be provided for the suture illustrated in FIG. 2 to pull the penile prosthesis cylinders 620, 625 into the neoglans 605.

The dual neophallus neotunica 600 may come in different sizes to meet different patients' anatomical needs. The neophallus neotunica may be constructed of biocompatible materials including silicone, polypropylene, PVDF or combinations thereof.

Single or dual penile implant surgical procedures occur at a final or near final stage of serial reconstructive procedures for cases of aphallia such as a female-to-male gender affirming surgery or surgery for congenitally deformed males (i.e., exstrophy). Thus, the neophallus and neoscrotum will have been previously created.

Proceeding at this stage for placement of the neophallus neotunica, the surgeon positions the patient in the dorsal lithotomy position. The genital area is surgically exposed, and an opening incision is made in the midline beneath the penoscrotal reconstruction and anterior to the anus, which provides bilateral access to the ischiopubic rami. Dissection continues on one lateral side for a single cylinder penile prosthesis or bilaterally for a two-cylinder penile prosthesis, exposing the ramus unilaterally or bilaterally, respectively, whereby the penile prosthesis will be surgically anchored.

Measurement lengths of the neophallus are determined, and the appropriately sized penile prosthesis cylinder(s) and a neophallus neotunica are then brought to the operative stage. A side table may then be used to assemble and prepare the penile prosthesis and the neophallus neotunica. In cases where the penile prosthesis is made available in the operating theater in sterile package separate from the neophallus neotunica, the penile prosthesis may be pulled into the neo-glans by a suture emanating from the penile prosthesis cylinder(s). The suture may then be passed through the neo-glans to further install the penile prosthesis in the neophallus neotunica. The suture is then available to pull the penile prosthesis ensheathed by the neophallus neotunica into the neophallus.

Two elastic bands may then be wrapped around the proximal firm end of the ensheathed inflatable penile prosthesis on each side of the exit tubing.

After this is done, the surgeon proceeds with insertion of the ensheathed penile prosthesis into the neophallus. The proximal end of the penile prosthesis is anchored to the ischiopubic structures, and the incision is closed.

1. A system for ensheathing at least one penile prosthesis cylinder in a neophallus comprising:
at least one penile prosthesis cylinder having a body portion and a distal end;
a socket for containing the distal end of the at least one penile prosthesis cylinder; and
a sheath for securing the body of the at least one penile prosthesis cylinder.

2. The system of claim 1, wherein the socket is in the shape of a glans.

3. The system of claim 1, wherein the socket is in the form of at least one socket catch for fastening the at least one penile prosthesis cylinder.

4. The system of claim 1, wherein the socket is ensheathed in a soft material approximating the feel of an anatomical glans.

5. The system of claim 1, wherein the sheath is a flexible cylinder.

6. The system of claim 1, wherein the sheath has a rough outer surface comprising ridges

7. The system of claim 1 wherein the sheath is comprised of a mesh.

8. The system of claim 1 wherein the sheath is comprised of an elastic material

9. The system of claim 1, wherein the socket is encased within the sheath.

10. The system of claim 1, wherein the socket is attached to the sheath by a joint.

11. The system of claim 1, further comprising elastic bands for securing the sheath to the at least one penile prosthesis cylinder.

12. A system providing changing a cylindrical shape of a penile prosthesis from flaccid to rigid and back again comprising:
an inner expansile cylinder;
an outer cylinder, wherein there is a space between the inner expansile cylinder and the outer cylinder;
an inflatable cylinder inside the inner expansile cylinder; and
columns of thin, overlapping disks included within the space between the inner expansile and outer cylinders.

13. The system of claim 12, wherein a soft matrix material suspends the thin, overlapping disks within the space between the inner expansile and outer cylinders.

14. The system of claim 12, wherein a loosely woven fabric structure suspends the thin, overlapping disks within the space between the inner expansile and outer cylinders.

15. A method for surgically installing a sheath in a neophallus, the method comprising:
making an opening incision in a patient;
dilating the neophallus;
installing the sheath by inserting the sheath into the neophallus; and
closing the incision.

16. The method of claim 15, further comprising cutting the sheath to size before inserting into the neophallus.

17. The method of claim 15, further comprising attaching a suture to the sheath, wherein the suture is pierced through the neophallus with a needle and used to pull the sheath into the neophallus.
